(19) United States
(12) Patent Application Publication
(10) Pub. No.: US 2019/0350791 A1
(43) Pub. Date: Nov. 21, 2019

(54) PATIENT COUCH WITH APPARATUS FOR REVERSIBLY RECEIVING A TRANSFER BOARD

(71) Applicant: Siemens Healthcare GmbH, Erlangen (DE)

(72) Inventors: Kirsten LUDWIG, Schwarzenbruck (DE); Markus PETSCH, Erlangen (DE)

(73) Assignee: Siemens Healthcare GmbH, Erlangen (DE)

(21) Appl. No.: 16/404,957

(22) Filed: May 7, 2019

(30) Foreign Application Priority Data
May 16, 2018 (DE) 102018207636.7

(51) Int. Cl. A61G 7/10; A61B 5/055; A61B 6/04

(52) U.S. Cl. CPC A61G 7/103; A61G 2210/50; A61B 6/0407; A61B 5/0555

(57) ABSTRACT

A patient couch of an imaging device includes an apparatus for reversible adjustment of the patient couch for reversibly receiving a transfer board. In an embodiment, the apparatus includes at least one guide track for guiding the transfer board. The patient couch further includes a first coupling element and the guide track includes a second coupling element, configured so as to functionally interact with the first coupling element, to produce a detachable connection between the patient couch and the guide track.

### PRIORITY STATEMENT

The present application hereby claims priority under 35 U.S.C. § 119 to German patent application number DE 102018207636.7 filed May 16, 2018, the entire contents of which are hereby incorporated herein by reference.

### FIELD

Embodiments of the invention generally relate to a patient couch of an imaging device with an apparatus for the reversible adjustment of the patient couch for reversibly receiving a transfer board, wherein the apparatus and the patient couch can be detachably connected to each other by coupling elements.

### BACKGROUND

During the course of an operative treatment of a sedated or anaesthetized patient, before, during and after the procedure it is frequently necessary to bring the patient to an imaging device, for example a magnetic resonance imaging device, in order to acquire corresponding scans, which are used to prepare or monitor the treatment. This transfer frequently takes place via a moveable patient-bearing couch, which has a transfer board, which is transferred from the patient-bearing couch to the patient couch of the imaging device.

One example of treatment, which requires image monitoring, is a neurosurgical procedure. During the procedure the patient rests on the transfer board, which is arranged on the neurosurgical operating table. In order to monitor the operation, the transfer board is now transferred from the operating table to the patient-bearing couch, which is used to transport the patient between the operating table and the patient couch of the imaging device and which is then moved to the patient couch of the magnetic resonance imaging device where the transfer board is transferred again. After acquiring the images, the transfer board is transferred to the patient bearing couch again.

A different example of treatment is a catheter ablation, which frequently occurs under X-ray monitoring. During the ablation process the transfer board is arranged on the couch of a C-arm X-ray apparatus. Guidance of the catheter can be followed during the course of the X-ray monitoring. In order to monitor the success of the ablation, the transfer board is again transferred to the patient-bearing couch and from this to the patient couch of the imaging device, in other words, for example the MR device, where the corresponding images are then acquired.

Alternatively, a mobile patient couch, which can be decoupled from the imaging device, can also be used, so the transfer board can be transferred directly from an operating table to the patient couch and the patient couch itself is used for the transfer of the patient.

The patient couch conventionally holds device(s) ready for receiving the transfer board for the transfer via transfer board, and these allow the transfer board to be transferred, in contrast to a standard patient couch, which holds an essentially flat surface ready on which the patient themselves is placed horizontally.

### SUMMARY

At least one embodiment of the present invention provides a patient couch of an imaging apparatus, which allows versatile use even in operative situations.

Embodiments relate to an inventive patient couch of an imaging device with an apparatus for the reversible adjustment of the patient couch, as well as an inventive patient couch system; and these and other embodiments are described in the claims. Advantageous embodiments and those that are regarded as being inventive in their own right are the subject matter of the claims and the following description.

At least one embodiment of a patient couch of an imaging device, with an apparatus for the reversible adjustment of the patient couch for reversibly receiving a transfer board, comprises:

at least one guide track for guiding the transfer board,

wherein the patient couch has a first coupling element and wherein the guide track has a second coupling element, which is configured in such a way as to functionally interact with the first coupling element, so a detachable connection can be produced between the patient couch and the guide track.

According to at least one embodiment of the invention, the apparatus includes at least one guide track on which the transfer board can be guided during receiving of the transfer board by the patient couch and which has a contact region which during guidance of the transfer board is in contact with the transfer board. The transfer board can be pulled or pushed, for example, over the contact region. The contact region of the guide track for the transfer board is arranged in a horizontal plane, so the transfer board can be moved essentially horizontally on the patient couch. An essentially sliding movement, in other words a uniform and constant movement of the transfer board, is enabled. An essentially sliding movement ensures optimally smooth and gentle receiving of the transfer board and of the patient.

According to at least one embodiment of the invention, the patient couch has a first coupling element and the guide track a second coupling element, wherein the second coupling element is configured to functionally interact with the first coupling element. The second coupling element can accordingly be coupled to the first coupling element. The first and the second coupling elements can, for example, be adjusted to each other in such a way that on adjustment of the patient couch they can be introduced into each other in a manner similar to a tongue and groove connection. For example, the coupling elements can on the one hand also comprise different recesses that are adapted to each other, and on the other hand projections, for example cylindrical ones, which are introduced into each other on adjustment of the patient couch. For example, a hook and a suspended portion or a bar and a counterpiece provided for this can also be provided on the guide track and the patient couch, and these couple the guide track and the patient couch to each other. The coupling elements can also have a different design, however.

At least one embodiment of the invention also relates to a patient couch system of a magnetic resonance tomography imaging device, comprising

a patient couch of one of the previously described advantageous embodiments, including an apparatus for reversible adjustment of the patient couch;

a local coil for the magnetic resonance examination of a patient; and

an attachment belt, which is designed for fixing the local coil on the patient, having a third coupling element, which is configured in such a way as to functionally interact with the first coupling element of the patient couch, so a detachable connection can be produced between the attachment belt and the patient couch.

At least one embodiment of the invention also relates to patient couch of an imaging device comprising:
an apparatus for reversible adjustment of the patient couch for reversibly receiving a transfer board, the apparatus including
   at least one guide track to guide the transfer board; and
a first coupling element, wherein the at least one guide track of the apparatus includes a second coupling element, configured to functionally interact with the first coupling element, to produce a detachable between the patient couch and the at least one guide track.

### BRIEF DESCRIPTION OF THE DRAWINGS

Example embodiments of the invention will be explained in more detail below using figures. In the drawings:

FIG. 1 shows a top view of an inventive patient couch having an apparatus for reversible adjustment of the patient couch according to one embodiment,

FIG. 2 shows a schematic cross-section through part of an inventive patient couch having an apparatus for reversible adjustment of the patient couch according to one embodiment, and

FIG. 3 shows a longitudinal section through part of an inventive patient couch having an apparatus for reversible adjustment of the patient couch with a receiving space for an optional imaging element.

### DETAILED DESCRIPTION OF THE EXAMPLE EMBODIMENTS

In the following, embodiments of the invention are described in detail with reference to the accompanying drawings. It is to be understood that the following description of the embodiments is given only for the purpose of illustration and is not to be taken in a limiting sense. It should be noted that the drawings are to be regarded as being schematic representations only, and elements in the drawings are not necessarily to scale with each other. Rather, the representation of the various elements is chosen such that their function and general purpose become apparent to a person skilled in the art.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of a patient couch of an imaging device, with an apparatus for the reversible adjustment of the patient couch for reversibly receiving a transfer board, comprises:

at least one guide track for guiding the transfer board,

wherein the patient couch has a first coupling element and wherein the guide track has a second coupling element, which is configured in such a way as to functionally interact with the first coupling element, so a detachable connection can be produced between the patient couch and the guide track.

According to at least one embodiment of the invention, it was recognized that a permanent adjustment, in other words, a change in a standard patient couch that cannot be reversed again using simple device(s), and/or a limitation of a functionality compared to a standard patient couch without receiving apparatus for a transfer board, is frequently connected with the possibility of receiving of a transfer board by a patient couch. For example, even in normal imaging situations, which do not require the use of a transfer board, use of a transfer board then becomes imperative and/or the functionality is limited as, for example, the use of optional imaging elements is limited. This affects, for instance, the use of a local spine coil, which during magnetic resonance tomography imaging is arranged underneath a patient to be examined is prevented.

Advantageously, at least one embodiment of the invention allows a reversible adjustment of a patient couch of an imaging device, wherein the functionality of the patient couch can be easily established again without adjustment by the apparatus.

The imaging device can be selected, for example, from the group comprising a magnetic resonance tomograph device, a computed tomography device, a PET-CT device, a SPECT-CT device, a SPECT device, an X-ray apparatus, a C-arm X-ray apparatus and combinations thereof.

A reversible adjustment of the patient couch can be taken to mean that the adjustment of the patient couch by the apparatus can be reversed, in particular non-destructively, so the functionality of the patient couch before adjustment by the apparatus can be established again without limitations and the apparatus is available again for a repeated adjustment of the patient couch.

The patient couch can therefore be temporarily adjusted or mechanically expanded by the apparatus, so a transfer board can be temporarily received or transferred, for example from a different patient-bearing couch, by the patient couch as soon as transfer of the patient via a transfer board is necessary for an examination of a patient with the imaging device. The mechanical expansion of the patient couch by the apparatus can also be undone again, however, if, for example, no transfer board is necessary for the examination of a patient in the imaging device, without the functionality of the patient couch being impaired by the possibility of adjustment of the patient couch.

The apparatus is provided to enable gentle and smooth receiving of a patient lying on a transfer board, for example in order to monitor operation in the imaging device. Without equipping of the patient couch with the apparatus there is no possibility of such simple, smooth receiving of a patient by the patient couch via transfer board.

In this connection a transfer board should in particular be taken to mean a board for supporting and/or positioning the patient, wherein the transfer board can be changed between different patient-supporting apparatuses and/or operating tables. Receiving of a transfer board by the patient couch means, for example, that a transfer board can be transferred from a further patient-bearing couch to the patient couch as the transfer board can be pushed or pulled from the patient-bearing couch onto the patient couch, for example along the longitudinal axis or the transverse axis of the patient couch and, following an examination with the imaging device, can be likewise transferred from the patient couch onto the patient-bearing couch again. Receiving can be carried out manually by operating personnel who move the transfer board with muscle power, in other words pull or push, but also with the aid of a motor.

According to at least one embodiment of the invention, the apparatus includes at least one guide track on which the transfer board can be guided during receiving of the transfer board by the patient couch and which has a contact region which during guidance of the transfer board is in contact with the transfer board. The transfer board can be pulled or pushed, for example, over the contact region. The contact region of the guide track for the transfer board is arranged in a horizontal plane, so the transfer board can be moved essentially horizontally on the patient couch. An essentially sliding movement, in other words a uniform and constant movement of the transfer board, is enabled. An essentially sliding movement ensures optimally smooth and gentle receiving of the transfer board and of the patient.

The guide track can in particular also predefine a preferred direction for receiving and relinquishing the transfer board, along which the transfer board is preferably guided, in other words along which the transfer board can be preferably moved. The guide track can have for example an elongate construction. It can be arranged along the longitudinal axis of the patient couch. As a result of the design of the guide track or the contact surface between guide track and the transfer board, a preferred direction can be predefined for receiving of the transfer board along this longitudinal axis. For example, the guide track has a smooth rail along which the transfer board can be moved in a sliding manner. For example, the guide track also has rollers with a designated axis of rotation, so the guide track is in contact with the rollers and on advancement of the transfer board perpendicular to the axis of rotation, the rolling movement of the rollers enables guidance of the transfer board perpendicular to the axis of rotation. The guide track can also be designed in a different way, however.

The guide track can be produced from one component or also be assembled from several components. The construction of the guide track can be configured such that surface regions of the guide track rest on the patient couch or, for example, laterally adjoin a lateral part or a housing part of the patient couch and are thereby adjusted to the construction of the patient couch. A stable arrangement of the guide track on the patient couch can be enabled thereby.

In particular, the apparatus can also have more than one guide track. The apparatus can have, for example, at least two guide tracks, wherein one guide track is arranged on the left longitudinal side and one is arranged on the right longitudinal side of the patient couch respectively and they are arranged so as to adjoin the patient couch from the inside. This enables simple and stable guidance of the transfer board along the longitudinal axis of the patient couch. However, other embodiments can also be provided, for example a single guide track which is arranged centrally in the patient couch, or a plurality of guide tracks arranged along the transverse axis, wherein the number, design and arrangement of the guide track or the guide tracks is selected in such a way that as to enable stable guidance and movement of the transfer board on the patient couch on receiving.

The guide track can comprise, for example, a fiber composite, plastics material or metal or a combination thereof. Preferably, the guide track comprises a material, which is non-conductive and non-magnetic, for example a fiber composite or plastics material.

According to at least one embodiment of the invention, the patient couch has a first coupling element and the guide track a second coupling element, wherein the second coupling element is configured to functionally interact with the first coupling element. The second coupling element can accordingly be coupled to the first coupling element. The first and the second coupling elements can, for example, be adjusted to each other in such a way that on adjustment of the patient couch they can be introduced into each other in a manner similar to a tongue and groove connection. For example, the coupling elements can on the one hand also comprise different recesses that are adapted to each other, and on the other hand projections, for example cylindrical ones, which are introduced into each other on adjustment of the patient couch. For example, a hook and a suspended portion or a bar and a counterpiece provided for this can also be provided on the guide track and the patient couch, and these couple the guide track and the patient couch to each other. The coupling elements can also have a different design, however.

In particular, the functional interaction between the first and the second coupling elements leads to a detachable connection of the guide track with the patient couch. Here, detachable can be taken to mean, in particular, that the connection can be reversible and, preferably, can be produced and also be cancelled again without an additional tool and preferably manually. In particular, the connection can be produced and detached again several times and repeatedly. The connection can in particular be non-destructively detached. Advantageously, the patient couch can thereby be easily adjusted in a time-efficient manner and without third device(s) being necessary.

The second coupling element of the guide track and the further parts of the guide track can be designed as one component or as an assembled component. Similarly, the first coupling element can be designed as a separate component and be assembled with the further parts of the patient couch or be integrated directly in the material of the patient couch, for example as a recess.

Preferably, the first coupling element is arranged on the patient couch in such a way that the functionality of the patient couch is not affected, for example by an available horizontal surface on the patient couch being limited by the coupling element. For example, the first coupling element is arranged on an outer edge, on outer housing parts or a side wall of the patient couch.

Advantageously, the patient couch can be reversibly adjusted by the apparatus and be mechanically expanded to temporarily receive a transfer board, wherein the functionality of the patient couch can be easily re-established without adjustment by the apparatus. Advantageously, a detachable connection between the apparatus and the patient couch can be produced, so the patient couch can be easily adjusted in a time-efficient manner and without third device(s) being necessary.

In an advantageous embodiment of the invention, it is provided that the guide track has a plurality of rollers for guiding the transfer board along the guide track.

Preferably, the rollers are attached to a side opposing the bearing surface of the patient couch and are in contact with the transfer board on guidance of the transfer board during the receiving process. The contact surface between the guide track and the transfer board in this embodiment accordingly comprises the bearing surface of the transfer board on the rollers. The rollers enable an essentially sliding movement of the transfer board due to a rolling movement of the rollers on displacement of the transfer board, for example by pushing or pulling the transfer board, in the direction of the rolling movement. Advantageously, optimally smooth and gentle receiving of the transfer board and of the patient is possible thereby. In addition, simple and low-friction guidance of the transfer board can be achieved thereby.

The rollers can have a designated axis of rotation and therewith, with a parallel arrangement of the axes of rotation of the plurality of rollers on the guide track, a preferred direction for receiving the transfer board perpendicular to the axes of rotation. The designated preferred direction can enable reliable displacement and guidance of the transfer board on the patient couch. However, the use of rollers without designated axis of rotation is also possible.

In a further advantageous embodiment of the invention, the first coupling element comprises an indentation in the form of a narrow channel along the longitudinal axis of the patient couch.

The narrow channel can correspond to a groove-like indentation in the material of the patient couch. An embodiment of this kind of the first coupling element enables a compact arrangement of the first coupling element on the patient couch. For example, the narrow channel can be arranged along an outer edge or in a lateral housing part or a side wall of the patient couch and extend over the entire longitudinal axis of the patient couch and therewith also provide a coupling possibility for the apparatus over the entire longitudinal axis of the patient couch. In other possible embodiments the narrow channel can also be arranged in a different way. The narrow channel can have for example a U-shaped profile or even a more complex and asymmetric profile in cross-section.

Furthermore, in a further advantageous embodiment of the invention, the second coupling element comprises a projecting section of the guide track, which is configured in such a way as to be introduced into the indentation of the first coupling element, so a displacement of the guide track in the horizontal plane transverse to the indentation is prevented.

The projecting section of the guide track can be introduced into the indentation of the first coupling element, so essentially a tongue and groove-like connection can be produced between the patient couch and the guide track. This provides a time-efficient and simple possibility for producing a detachable connection and for ensuring an arrangement of the guide track on the patient couch that is secured against displacement. Advantageously, no additional device(s) or tool are/is required for producing the detachable connection. The introduction can comprise a simple, vertical insertion or else the projecting section can be introduced, for example, only at a particular angle into the guide track and with a subsequent rotational movement be brought into the intended position on the patient couch. The projecting section can also be configured to be changeable in shape, for example be essentially elastically bendable, and the introduction can comprise temporary bending of the projecting section during introduction.

In an advantageous embodiment of the invention, the indentation of the first coupling element has an overhang. In addition, a nose is formed on the projecting section of the second coupling element, which, following the adjustment of the patient couch, engages under the overhang, so the guide track is secured against unintentional release from the patient couch.

The second coupling element is accordingly latched in the first coupling element in this embodiment. In particular, the connection between the guide track and the patient couch can therefore be detached again only by a rotational movement, in other words a rotation of the guide track along the longitudinal axis and away from the patient couch, so simple, vertical lifting out and unintentional lifting-out of the guide track from the patient couch is impossible. A secure arrangement of the guide track and therewith secure guidance of the transfer board can be advantageously enabled thereby.

The introduction of the second coupling element into the indentation and under the overhang of the first coupling element can comprise for example through an elastic deformation of the projecting element during coupling or similarly a rotational movement, so the second coupling element can be introduced into the indentation only at a particular angle relative to a horizontal plane and the guide track is then brought by way of a rotational movement into the intended position on the patient couch, for example such that a surface region of the guide track rests on the patient couch. The nose of the projecting element engages under the overhang in the process and secures the guide track against being vertically lifted out. Advantageously, the guide track is securely coupled to the patient couch, it being possible for the coupling to occur manually.

Furthermore, it is proposed that in a further advantageous embodiment of the invention, the guide track also has a first fixing element, which is configured to fix the guide track to the patient couch with a second fixing element provided for this purpose on the patient couch, wherein the connection between the guide track and the patient couch can only be detached after cancelling the fixing.

The fixing elements can be used in particular for an additional fixing and securing of the guide track on the patient couch, so the functional interaction between the coupling elements can also be cancelled only after detaching the fixing and/or can be used for fixing the position of the guide track relative to the patient couch, so the guide track cannot be displaced in an operating state coupled to the patient couch. In particular, fixing can be cancelled manually and preferably without third device(s). The fixing elements can have a design similar to a tongue and groove system, that is, an indentation or recess on the one hand and a projection adapted to the indentation or recess on the other hand, which can be introduced into each other for additional fixing of the guide track to the patient couch. Other embodiments can comprise for example a bar and a corresponding counterpiece, a folding bar, a lock or other fixing elements suitable for the purpose, which also fix the guide track to the patient couch, it being possible to manually cancel the fixing again. Advantageously, improved secure arrangement of the guide track on the patient couch can be achieved thereby. The fixing element provided on the patient couch can be arranged for example on a foot-end or head-end housing part of the patient couch.

One advantageous embodiment of the invention provides that the patient couch and the apparatus are configured in such a way that, following the adjustment of the patient couch with the apparatus, a cavity is formed between the patient couch and the guide track for receiving signal lines.

For example, the guide track has a recess on a side of the guide track facing the patient couch and on surface regions adjoining the recess, which, after the patient couch has been fitted with the apparatus, rest on the patient couch or, for example, also laterally adjoin a lateral part or housing part of the patient couch, so the recess, following adjustment of the patient couch, delimited firstly by the guide track and secondly by the patient couch, forms a cavity, which is capable of accommodating signal lines. With an example arrangement of an elongate guide track along the longitudinal axis of the patient couch, this cavity can preferably also run along the longitudinal axis and thereby permit installation of signal lines along the longitudinal axis of the patient couch. For example, access to signal lines, which would be rendered difficult as a result of equipping of the patient couch with the apparatus due to partial covering of the access points or signal lines by the apparatus, can be facilitated thereby even following adjustment of the patient couch by the apparatus.

For example, hoses with a larger diameter than the signal lines themselves can also be conducted inside the cavity for easier handling, which hoses can in turn encompass the signal lines, so the signal lines can be handled more easily.

The signals, which are transported by the signal lines, can be for example signals relevant to the imaging mechanism, but also sound or image signals which are to be supplied to a patient, who is to be imaged by the imaging device, or for example also an emergency off signal, which can be triggered by the patient themselves.

Advantageously, the functionality of the patient couch with and without adjustment by the apparatus can be kept constant.

In an advantageous embodiment of the invention, the patient couch and the apparatus are configured in such a way that, following adjustment of the patient couch by the apparatus, a receiving space is formed between the guide track and the patient couch for an optional imaging element.

Following adjustment of the patient couch, in other words mechanical expansion by the inventive apparatus, the receiving space can be upwardly and laterally delimited, at least in sections, by the guide track. The optional imaging element can be then only partially or completely covered by the guide track. The guide track can also rest on the optional imaging element. The optional imaging element can also provide a supporting function for the apparatus.

For example, the optional imaging element is a local spine coil for a magnetic resonance tomography examination of a patient, which is arranged underneath the patient and which is placed or put on the essentially horizontal surface of the patient couch. The recess of the guide track and therewith the receiving space can accordingly be configured in such a way that, for example, sufficient space is available on the essentially horizontal surface of the patient couch, so the spine coil can be arranged on the patient couch at the same time as the apparatus for receiving the transfer board.

Advantageously, the patient couch can be adjusted to the imaging modalities. Advantageously, the same functionality of the patient couch with and without an adjustment can be ensured by an inventive apparatus thereby and a limitation of the imaging possibilities as a result of equipping the patient couch with an apparatus of his kind can be avoided.

Furthermore, one advantageous embodiment of the invention provides that the apparatus has right and left guide tracks along the longitudinal axis of the patient couch, and wherein each guide track is divided into at least two guide track parts that can be detachably connected together.

Handling of the guide track when the patient couch is equipped with the apparatus can be facilitated as a result of the arrangement of right and left guide tracks along the longitudinal axis of the patient couch and the division into at least two guide track parts, so the guide track parts, which are each configured for example to be shorter and therewith easier to handle than the guide track itself, can be individually coupled to the patient couch. The at least two guide track parts can then be detachably connected to form a guide track. Detachable can in particular be taken to mean reversibly connectable. Preferably, the connection is detachable without the use of an additional device(s) or tool, which is not directly arranged on the guide track or the patient couch, and can be manually detached instead. For example, a bar for folding or displacement is provided on the guide track parts, and this fastens the guide track parts to each other. Another possibility could comprise suspending a guide track part in a second guide track part, for example with a provided hook. A connection which can be detached using simple device(s) can improve the secure arrangement of the guide track and therewith reliable guidance and receiving of the transfer board.

At least one embodiment of the invention also relates to a patient couch system of a magnetic resonance tomography imaging device, comprising

a patient couch having an apparatus for reversible adjustment of the patient couch in one of the previously described advantageous embodiments,

a local coil for the magnetic resonance examination of a patient; and

an attachment belt, which is designed for fixing the local coil on the patient, having a third coupling element, which is configured in such a way as to functionally interact with the first coupling element of the patient couch, so a detachable connection can be produced between the attachment belt and the patient couch.

The third coupling element of the attachment belt can be configured similar to or so as to match the second coupling element of the guide track, so the attachment belt can be repeatedly reversibly connected and detached, and preferably without additional third device(s), as well as manually, to/from the patient couch. Preferably, the third coupling element is secured against vertical release. This can be achieved by design of the first and third coupling elements, for example by an overhang on the first coupling element and a nose formed on the third coupling element, which after coupling, engages under the overhang, or also by an additional element, which fixes the third coupling element or the attachment belt in the first coupling element or to the patient couch.

Advantageously, the magnetic resonance tomography imaging device can thereby also be used for examinations on patients in which the transfer of a patient via a transfer board onto the patient couch of the imaging device is necessary, as the patient couch can be reversibly and temporarily adjusted via the apparatus for receiving a transfer board.

Advantageously, the same coupling elements on the patient couch can also be used for the connection of local coils for an imaging examination.

Because the first coupling element is available for the reversible connection of the apparatus as well as for the reversible connection of attachment belt, versatile use of the patient couch is advantageously possible. In addition, compact attachment of the coupling elements can be ensured thereby.

An advantageous embodiment of the patient couch system provides that the first coupling element is configured such that the second coupling element and the third coupling element can be coupled to the first coupling element at the same time, so the attachment belt and the guide track of the apparatus can be detachably connected to the patient couch at the same time.

The second and the third coupling elements can be designed in such a way that they each use only a section of the first coupling element for producing the detachable connection between the guide track and the patient couch or between the attachment belt and the patient couch and apart from that, keep a different section of the first coupling element available for further connections. The patient couch can consequently be equipped with an inventive apparatus for reversibly receiving a transfer board and, similarly, attachment belts for fixing a local coil on the patient can be connected to the patient couch at the same time. The attachment belts and the apparatus can also be connected separately to the patient couch, however, in other words in particular in a staggered manner, and be used with the patient couch independently of each other.

Advantageously, the same functionality of the patient couch with and without an adjustment can be ensured by an inventive apparatus and a limitation of the imaging possibilities as a result of equipping the patient couch with an apparatus for reversibly receiving a transfer board can be avoided.

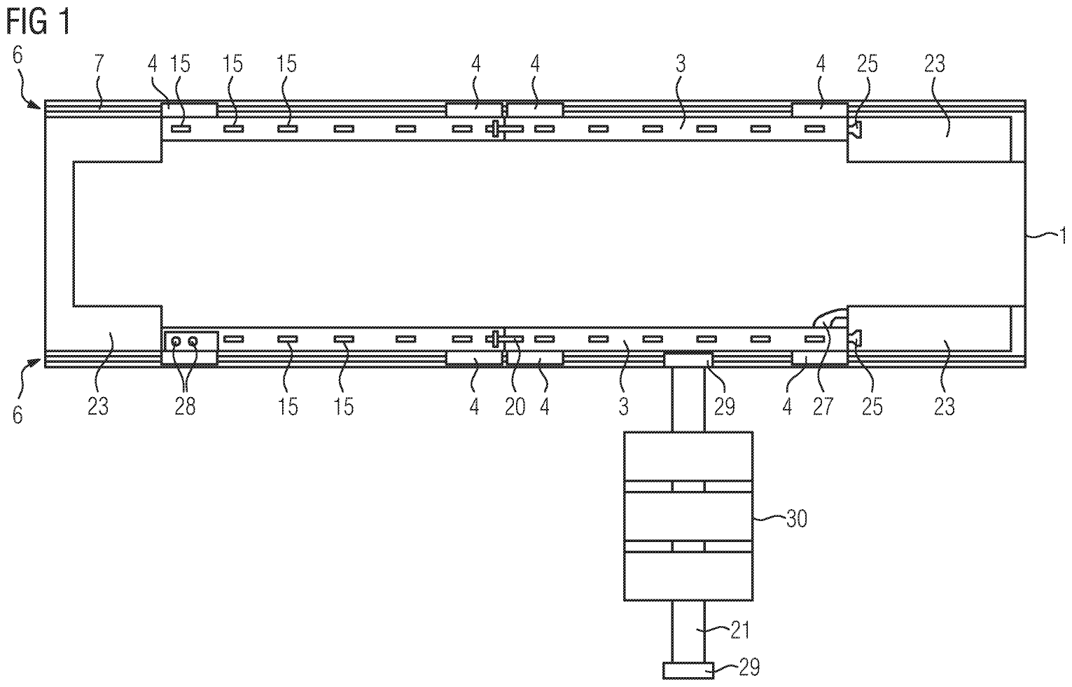

In FIG. 1 shows a schematic top view of an inventive patient couch (1) having an apparatus for reversible adjustment of the patient couch (1) according to one example embodiment and in an operating state in which receiving or transfer of a transfer board, for example from a further patient-bearing couch, is possible as a result of equipping the patient couch (1) with the apparatus.

Along the longitudinal axis at its right and left edges, for example at side walls, the patient couch (1) has one first coupling element (6), which comprises an indentation (7) in the form of a narrow channel and along the entire longitudinal axis of the patient couch (1) respectively. In this embodiment the apparatus for reversible adjustment of the patient couch (1) has two guide tracks (3), which are arranged so as to adjoin the left and right edges, for example side walls, and are arranged along the longitudinal axis of the patient couch (1) respectively. The guide tracks (3) interact by way of a plurality of coupling elements (4) arranged distributed on guide tracks (3) with the left first and the right first coupling elements (6) on the patient couch and are detachably connected by the coupling to the patient couch (1), for example as a projecting element (9) (not visible in this view) is formed on the second coupling element (4), and this can be introduced into the indentation (7) and thereby secures the guide track (3) against displacement perpendicular to the indentation (7) and detachably connects it to the patient couch (1). The connection can be reversibly produced and preferably without an additional tool and preferably manually and can also be cancelled again. Advantageously, the patient couch can be adjusted easily and in a time-efficient manner and without third device(s) being required.

The guide tracks (3) also have a plurality of rollers (15), which during receiving and guidance of the transfer board are in contact with the transfer board and enable an essentially sliding movement of the transfer board due to a rolling movement of the rollers (15) on displacement of the transfer board, for example by pushing or pulling the transfer board, in the direction of the rolling movement. In the illustrated arrangement the guide tracks (3) have a preferred direction for receiving and relinquishing the transfer board along the longitudinal axis of the patient couch (1).

In this example embodiment the guide tracks (3) are also divided along the longitudinal axis into two guide track parts respectively, and these are manually detachably connected together by a connecting mechanism (20), for example a bar. This division of the guide track into guide track parts that are smaller in their dimensions enables easier handling of the apparatus.

The coupling elements (4) of the guide tracks (3) each cover just one section of the first coupling elements (6), so the first coupling elements (6) are available for further elements to be coupled. For example, an attachment belt (21) for an optional local coil (30), which is used in the case of an examination with a magnetic resonance tomography imaging device and is fixed via the attachment belt (21) on a patient, can therefore be coupled by a third coupling element (29) to the first coupling elements (6) of the patient couch (1) and also at the same time be used with the apparatus for reversible adjustment for receiving a transfer board. In the illustrated view the optional local coil (30) and the attachment belt (21) with the third coupling element (29) is shown only schematically and in an operating state coupled only on one side and folded to the side. For an examination the local coil is positioned above the patient couch and above a patient positioned on the patient couch and is detachably connected on both sides with the attachment belt (21) by the third coupling elements (29) to the left and the right sides of the patient couch (1).

The guide tracks (3) are fixed on one side by additional fixing elements (25) to housing parts (23) of the patient couch (1). Projections formed on the guide track are as fixing elements (25) in this embodiment, and these engage in recesses provided on the patient couch. Only after detaching the fixing elements (25) can the guide track (3) be released from the patient couch (1).

Furthermore, one of the guide tracks (3) of the apparatus also has extension elements (27) and access points (28) for signal lines, which are guided along the guide track, for example in a cavity (17) (not visible here) between the guide track (3) and the patient couch (1), along the longitudinal axis, so easy access to the signal lines is possible even after the adjustment of the patient couch (1) by the apparatus.

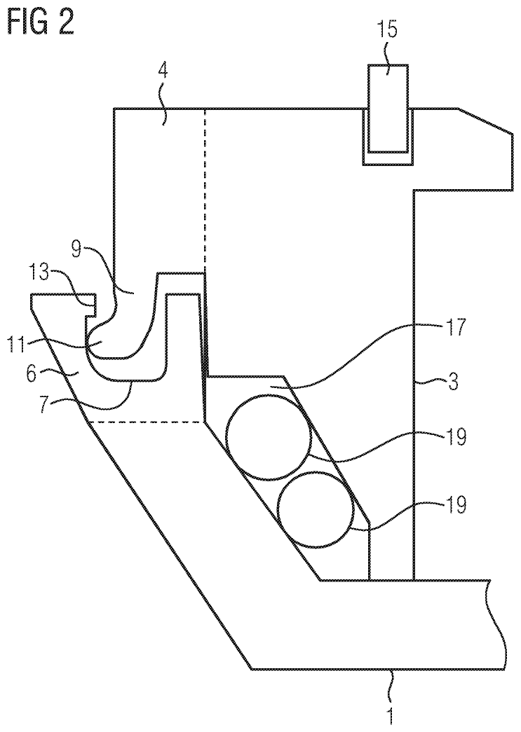

FIG. 2 shows a schematic cross-section through part of a patient couch (1) with an apparatus for reversible adjustment of the patient couch (1) according to one example embodiment. A cross-section through a guide track (3) of the apparatus is shown, which is arranged so as to laterally adjoin a side wall of the patient couch (1) and is arranged along the longitudinal axis of the patient couch (1). The guide track (3) of the apparatus is shaped in such a way that a surface region of the guide track (3) rests on the patient couch (1) and laterally adjoins a side wall of the patient couch (1).

The first coupling element (6), which is formed on the patient couch (1) on the side wall, in this example embodiment has an indentation (7) in the form of a narrow channel and an overhang (13). The coupling element (4) formed on the guide track (3) comprises a projecting element (9) on which a nose (11) is formed. In the illustrated operating state, that is after adjustment of the patient couch (1) by the apparatus, the projecting element (9) is introduced into the indentation (7) and the nose (11) engages under the overhang (13) of the first coupling element (6). In particular, the connection between the guide track (3) and the patient couch (1) can therefore only be detached again by a rotational movement, in other words a rotation of the guide track (3) along the longitudinal axis and away from the patient couch (1), so simple vertical lifting-out and unintentional release of the guide track (3) from the patient couch (1) is prevented.

Shown on the surface of the guide track (3) opposing the essentially horizontal surface of the patient couch (1) is one example roller (15) of the plurality of rollers (15), by way of which a transfer board can be guided during receiving on or relinquishing from the patient couch (1).

The structure of the guide track (3) also has a recess, so in the illustrated operating state of the patient couch mechanically expanded by the apparatus, a cavity (17) is formed between guide track (3) and patient couch (1). In this example embodiment the cavity (17) runs along the side wall of the patient couch (1) and is designed to receive signal lines, which can be installed along the longitudinal axis of the patient couch (1) and along the guide track (3). For example, access to signal lines can therefore be displaced and, after adjustment of the patient couch (1) by the apparatus, be more easily possible. In the illustrated example embodiment hoses (19) with a larger diameter than the signal lines themselves are provided inside the cavity (17) for the installation of the signal lines, and these grasp the signal lines for improved and easier handling, for example also more than one signal line per hose (19).

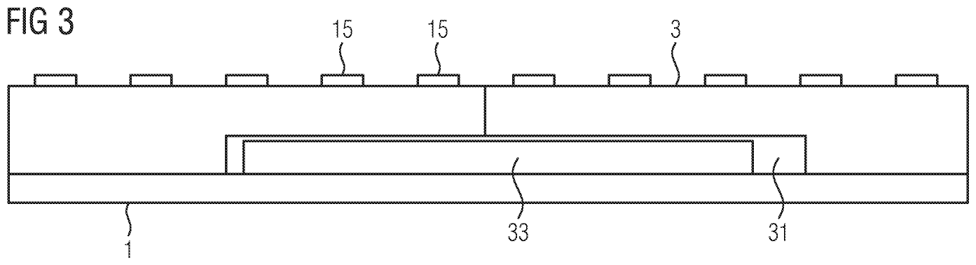

FIG. 3 shows a schematic longitudinal section through part of an inventive patient couch (1) after adjustment with an apparatus for reversibly receiving a transfer board, having a receiving space (31) for an optional imaging element (33). The longitudinal section shows in particular a section through a guide track (3), divided into two guide track parts, which is oriented along the longitudinal axis of the patient couch (1) and has surface regions facing the patient couch, which rest on the essentially horizontal region of the patient couch (1). A plurality of rollers (15) is formed on the surface of the guide track (3) opposing the essentially horizontal region of the patient couch (1), which is in contact with the transfer board on receiving of the transfer board and by way of which the transfer board can be guided along the longitudinal axis of the patient couch (1) on receiving and during displacement.

In addition, the guide track (3) has a recess, so after adjustment of the patient couch (1) with the apparatus a receiving space (31) is formed for an optional imaging element (33). The optional imaging element (33) can be for example only partially or also completely covered by the guide track (3). For example, the optional imaging element (33) is a spine coil for a magnetic resonance tomography examination, which is arranged underneath the patient and which for the examination is placed or put on the essentially flat surface of the patient couch (1). The recess of the guide track (3) and therewith the receiving space (31) is therefore preferably designed in such a way that for example sufficient space is available on the essentially horizontal surface of the patient couch (1), so the spine coil can be arranged on the patient couch at the same time as the apparatus for receiving the transfer board. Other optional imaging elements can comprise, for example, additional electronic devices. For example, the receiving space comprises a vertical height of 20 to 80 mm and a width along the transverse axis of the patient couch of 200 to 600 mm. In one specific embodiment the receiving space comprises at least a height of 41.5 mm, a length of 1,385 mm and a width of 490 mm. The receiving space can also have a different design, however.

Although the invention has been illustrated in more detail by the preferred example embodiment, it is not limited by the disclosed examples and a person skilled in the art can derive other variations herefrom without departing from the scope of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A patient couch of an imaging device, comprising:
an apparatus for reversible adjustment of the patient couch for reversibly receiving a transfer board, the apparatus including
at least one guide track to guide the transfer board; and
a first coupling element, wherein the at least one guide track of the apparatus includes a second coupling element, configured to functionally interact with the first coupling element, to produce a detachable between the patient couch and the at least one guide track.

2. The patient couch of claim 1, wherein the at least one guide track includes a plurality of rollers to guide the transfer board along the at least one guide track.

3. The patient couch of claim 1, wherein the first coupling element includes an indentation, in a form of a channel, along a longitudinal axis of the patient couch.

4. The patient couch of claim 3, wherein the second coupling element includes a projecting section of the at least one guide track, configured to be introducible into the indentation of the first coupling element, to prevent a displacement of the at least one guide track in a horizontal plane transverse to the indentation.

5. The patient couch of claim 4, wherein the indentation of the first coupling element includes an overhang and wherein a nose is formed on the projecting section of the second coupling element, which, after adjustment of the patient couch, is configured to engage under the overhang to secure the guide track against unintentional release from the patient couch.

6. The patient couch of claim 1, wherein the at least one guide track includes a first fixing element, a second fixing element being provided on the patient couch, configured to fix the at least one guide track to the patient couch, wherein a connection between the at least one guide track and the patient couch is detachable only after cancelling fixing via at least one of the first fixing element and the second fixing element.

7. The patient couch of claim 1, wherein the patient couch and the apparatus are configured such that, after adjustment of the patient couch with the apparatus, a cavity is formed between the patient couch and the at least one guide track for receiving signal lines.

8. The patient couch of claim 1, wherein the patient couch and the apparatus are configured such, after adjustment of the patient couch by the apparatus, a receiving space is formed between the at least one guide track and the patient couch for an optional imaging element.

9. The patient couch of claim 1, wherein the at least one guide track includes at least one right guide track and at least one left guide track, along a longitudinal axis of the patient couch, and wherein each of the at least one right guide track and at least one left guide track is divided into at least two guide track parts, detachably connected to each other.

10. A patient couch system of a magnetic resonance tomography imaging device, comprising:
the patient couch of claim 1, including the apparatus for reversible adjustment of the patient couch;
a local coil for magnetic resonance examination of a patient; and
an attachment belt, designed to fix the local coil on the patient, including a third coupling element, configured to functionally interact with the first coupling element of the patient couch, to provide a detachable connection between the attachment belt and the patient couch.

11. The patient couch system of claim 10, wherein the second coupling element and the third coupling element are simultaneously coupleable to the first coupling element, rending the attachment belt and the at least one guide track of the apparatus to be simultaneously detachably connected to the patient couch.

12. The patient couch of claim 2, wherein the first coupling element includes an indentation, in a form of a channel, along a longitudinal axis of the patient couch.

13. The patient couch of claim 12, wherein the second coupling element includes a projecting section of the at least one guide track, configured to be introducible into the indentation of the first coupling element, to prevent a displacement of the at least one guide track in a horizontal plane transverse to the indentation.

14. The patient couch of claim 13, wherein the indentation of the first coupling element includes an overhang and wherein a nose is formed on the projecting section of the second coupling element, which, after adjustment of the patient couch, is configured to engage under the overhang to secure the guide track against unintentional release from the patient couch.

15. The patient couch of claim 2, wherein the at least one guide track includes a first fixing element, a second fixing element being provided on the patient couch, configured to fix the at least one guide track to the patient couch, wherein a connection between the at least one guide track and the patient couch is detachable only after cancelling fixing via at least one of the first fixing element and the second fixing element.

16. The patient couch of claim 2, wherein the at least one guide track includes at least one right guide track and at least one left guide track, along a longitudinal axis of the patient couch, and wherein each of the at least one right guide track and at least one left guide track is divided into at least two guide track parts, detachably connected to each other.

17. The patient couch system of claim 10, wherein the at least one guide track includes a plurality of rollers to guide the transfer board along the at least one guide track.

18. The patient couch system of claim 10, wherein the first coupling element includes an indentation, in a form of a channel, along a longitudinal axis of the patient couch.

19. The patient couch system of claim 18, wherein the second coupling element includes a projecting section of the at least one guide track, configured to be introducible into the indentation of the first coupling element, to prevent a displacement of the at least one guide track in a horizontal plane transverse to the indentation.

20. The patient couch system of claim 19, wherein the indentation of the first coupling element includes an overhang and wherein a nose is formed on the projecting section of the second coupling element, which, after adjustment of the patient couch, is configured to engage under the overhang to secure the guide track against unintentional release from the patient couch.
